(19) United States
(12) Patent Application Publication
(10) Pub. No.: US 2014/0099682 A1
(43) Pub. Date: Apr. 10, 2014

(54) Enzymatic Synthesis of Optically Active Chiral Amines

(71) Applicants: Arumugam Govind Swaminathan, Mumbai (IN); Sunil Vaman Joshi, Maharashtra (IN)

(72) Inventors: Arumugam Govind Swaminathan, Mumbai (IN); Sunil Vaman Joshi, Maharashtra (IN)

(73) Assignee: EMBIO LIMITED, Powai, Mumbai (IN)

(21) Appl. No.: 14/008,725

(22) PCT Filed: Aug. 16, 2012
(86) PCT No.: PCT/IB12/54176

§ 371 Date: Dec. 18, 2013

(30) Foreign Application Priority Data
Aug. 16, 2011 (IN) 2312/MUM/2011

(51) Int. Cl. C12P 13/00

(52) U.S. Cl. CPC C12P 13/001
USPC 435/128

(57) ABSTRACT

The present invention relates to method of production of optically active chiral amine from alpha hydroxy ketone using enzyme transaminase as the biocatalyst. In particular the present invention relates to production of (1R, 2S)-Norephedrine and its salts from R-Phenylacetylcarbinol (R-PAC) by employing S-transaminase as the biocatalyst and Isopropylamine as the amine donor.

### FIELD OF THE INVENTION

The present invention is generally related to production of optically active chiral amine from alpha hydroxy ketone using enzyme transaminase as the biocatalyst. More particularly the present invention relates to production of (1R, 2S)-Norephedrine and its salts from R-Phenylacetylcarbinol (R-PAC) by employing S-transaminase as the biocatalyst and Isopropylamine as the amine donor.

### BACKGROUND OF THE INVENTION

Chiral amine plays an important role in the pharmaceutical and chemical industry. Chiral amines in general are frequently used as a resolving agents or intermediates or synthons for the preparation of various physiologically, for instance pharmaceutically active substances. In a great number of the various applications of chiral amines, only one particular optically active form, either the (R) or the (S) enantiomer has the desired physiological activity. Thus, there is a clear need to provide processes for the preparation of chiral amines in an optically active form.

Norephedrine or 2-amino-1-phenyl-1-propanol is a naturally occurring alkaloid found in Chinese herb ‘Ma Huang’ or Ephedra, also an optically active amine It is isolated from the herb along with 1-ephedrine and other alkaloids. Apart from the natural source, it can be synthesized by chemical methods. Norephedrine can be synthesized chemically by catalytic reductive amination, catalytic hydrogenation etc. One of the serious drawbacks associated with the chemical synthesis is that it does not provide diastereoselectivity, hence an equal quantity of diastereomer is obtained.

Prior art includes various synthetic methods for the preparation of 1-norephedrine such as

1. By resolution of dl-phenylpropanolamine

Some of the relevant patents are German Patents 2,258,410 (1973); 2,304,055 (1974) and 2,258,410 (1974) and British Patent 1,385,490 (1975) disclose resolution of dl-phenylpropanolamine employing thiazolidinecarboxylic acids. German Patent 2,258,507 (1976) discloses resolution of dl-phenylpropanolamine using pantoic acid. German patents 2,854,069 (1979) and 2,854,070 (1979) demonstrate use of maleamides of d- and 1-norpseudoephedrinein resolving dl-phenylpropanolamine Japanese Patent 4530 (1955) discloses resolution of dl-phenylpropanolamine using (2R, 3R)-2,3-dimethoxy succinic acid. JP-A 51/98231 also discloses resolution method.

The drawback associated with this prior art is poor yields (lack of diastereoselectivity), cost and difficulty of recovering these resolving agents.

2. Reductive amination of 1-1-hydroxy-1-phenyl-2-propanone

Some of the relevant patents are German Patents 588,880 (1933); 587,586 (1933); 599,433 (1934); 1,014,553 (1957); British Patents 365,535 (1930); 365,541 (1930); Indian Patent IN172970 (1994); EP 1142864

3. Reduction of derivatives of 1-1-phenyl-1-hydroxy-2-propanone

Reduction of 1-1-phenyl-1-hydroxy-2-propanone derivatives like oxime, hydrazones, N-benzylimine has been reported in British Patents 365,535 (1930); German patent 1,014,553 (1957), O. C. Kreutz; P. J. S. Moran and J. A. R. Rodrigues, Tetrahedron: Asymmetry 8, 2649-2653 (1997). Gaseous and liquid effluent generation and recyclability of catalyst are major concerns whereas EP2055379 overcomes some of these problems and reports a diastereomeric purity >97%.

The starting material, 1-1-hydroxy-1phenyl-2-propanone, however being an alpha-ketol is sensitive to extreme temperature and pH conditions leading to racemisation and isomerisation.

4. Resolution of 2-amino-1-phenyl-1-propanone followed by reduction

Some of the prior art pertaining to the given technique are German patent 639,129 (1936); Japanese Patent JP 63091352 (1988) and literatures like H. Takamatsu, J. Pharm. Soc. Japan 76, 1219-1222 (1956) and B. D. Berrang, A. H. Lewin, F. I. Carroll, J. Org. Chem. 47, 2643-2647(1982); F. Skita, F. Keil, E. Baesler, Chem. Ber. 66, 858 (1932).

The major drawback with respect to the cited prior art is that the starting material is unstable as a base, and resolution efficiency is poor and overall yield of the optically pure antipodes is very low. Furthermore, the catalytic hydrogenation of 2-amino-1-phenyl-1-propanone as described in, does not give exclusively erythro-product which is very essential for overall efficiency of the process.

### 5. Asymmetric Reduction

Jpn. Kokai Tokkyo Koho JP 0504948 [93,04948] (1993) patent describes a method in which alpha-isonitrosopropiophenone is asymmetrically hydrogenated in the presence of chiral substituted ferrocene catalysts. However this method also does not give a high diastereomeric and enantiomeric excess of one enantiomer of phenylpropanolamine over other and hence was not satisfactory.

### 6. Chiral Precursors

Yet another approach is a stereospecific synthesis of 1-erythro-2-amino-1-phenyl-1-propanol from chiral precursors (T. F. Buckley; H. Rapoport, J. Am. Chem. Soc. 103, 6157-6163 (1981); K. Koga; H. Matsou and S. Yamada, Chem. Pharm. Bull. 14, 243-246 (1966); W. R. Jackson; H. A. Jacobs; G. S. Jayatilake; B. M. Matthews and K. C. Watson Aust. J. Chem. 43, 2045 (1990)) or by use of chiral auxiliaries. (W. Oppolzer; O. Tamura; G. Surendrababu and M. Signer, J. Am. Chem. Soc. 114, 5900 (1992)).

In addition to the above described methods various other methods have been described by D. Enders; H. Lotter; N. Maigrot; J. P. Mazaleyrat and Z. Welvart, Nouv. J. Chem. 8, 747-750 (1984), and in Jpn. Kokai Tokkyo Koho JP 10 45688 (1998) in which alpha-isonitrosopropiophenone was either hydrogenated in the presence of hydrogenations having chiral ligands or reduced with borohydride complexes of 1,2-amino alcohol chiral auxiliaries.

Consequently, review of prior art methods based on synthetic chemistry, shows that all the above stated methods suffer from at least one of the following drawbacks such as cost and recyclability of hydrogenation catalyst, cost and recyclability of resolving agents, poor diastereo- and enantioselectivity in reductions, cost and availability of chiral precursors or chiral auxiliaries, cost and availability of chiral catalysts, generation of gaseous, liquid and solid effluents which may be hazardous.

In the view of the mentioned drawbacks in the prior art, there is an ongoing need to develop a process for the preparation of 1-erythro-2-amino-1-phenyl-1-propanol (1-Norephedrine) that bypasses the above limitations and is more efficient in terms of yield and resolution and at the same time is cost-effective for which an enzymatic approach would be the answer to the above mentioned problems.

### SUMMARY OF THE INVENTION

It is an object of the present invention to provide method of production of optically active chiral amine from alpha hydroxy ketone using enzyme transaminase as the biocatalyst. In particular the present invention relates to production of (1R, 2S)-Norephedrine and its salts from R-Phenylacetylcarbinol (R-PAC) by employing S-transaminase as the biocatalyst and Isopropylamine as the amine donor.

Accordingly, the present invention provides process of preparing optically active chiral amine comprising the following steps:
a) Providing an amino acceptor or keto substrate selected from a series of alpha hydroxy ketone and an amino donor
b) Reacting the keto substrate and the amino donor with a transaminase, in particular (R) or (S)-selective transaminase and
c) Finally obtaining the desired optically active chiral amine and a ketone by-product.

Thus, the present invention provides a process for the synthesis of optically active chiral amines by using at least one transaminase for the transamination of an amino group from an amino donor to a keto substrate acting as amino acceptor, thereby forming the desired product. Therefore, the present invention features an enzymatic method of producing optically active chiral amines by utilizing transaminase or aminotransaminase enzyme in the presence of defined amino donor.

### DETAILED DESCRIPTION OF THE INVENTION

The present invention relates to an improved method of producing optically active amine product using alpha hydroxy ketone as the substrate in the presence of an enzyme and an amino donor. In particular the present invention provides a process of preparation of an optically active amine comprising:
a) Providing an amino acceptor or keto substrate selected from a series of alpha hydroxy ketone and an amino donor
b) Reacting the keto substrate and the amino donor with a transaminase, in particular (R) or (S)-selective transaminase and
c) Finally obtaining the desired optically active chiral amine and a ketone by-product.

In order to more clearly and concisely describe and point out the subject matter of the claimed invention, the following definitions are provided for specific terms, which are used in the following written description.

“Transaminase” and “Aminotransferase” are used interchangeably herein to refer to a polypeptide having an enzymatic capability of transferring an amino group (NH₂), a pair of electrons, and a proton from a primary amine to a carbonyl group (C═O) of an acceptor molecule. Transaminases as used herein include naturally occurring (wild type) transaminase as well as non-naturally occurring engineered polypeptides generated by human manipulation.

“Keto substrate”, “Keto” “Ketone” and “Amino acceptor” are used interchangeably herein to refer to a carbonyl (keto, or ketone) compound which accepts an amino group from a donor amine.

“Amino donor”, “Amine donor” and “donor amine” are used interchangeably herein to refer to any amino acid or amine that will react with a transaminase and a ketone, to produce desired amine product and a ketone by product.

“Pyridoxal-phosphate”, “PLP”, “pyridoxal-5′-phosphate”, “PYP”, and “P5P” are used interchangeably herein to refer to the compound that acts as a coenzyme in transaminase reactions. In transamination reactions using transaminase enzymes, the amine group of the amino donor is transferred to the coenzyme to produce a keto byproduct, while pyridoxal-5′-phosphate is converted to pyridoxamine phosphate. Pyridoxal-5′-phosphate is regenerated by reaction with a different keto compound (the amino acceptor). The transfer of the amine group from pyridoxamine phosphate to the amino acceptor produces a chiral amine and regenerates the coenzyme. In some embodiments, the pyridoxal-5′-phosphate can be replaced by other members of the vitamin B₆ family, including pyridoxine (PN), pyridoxal (PL), pyridoxamine (PM), and their phosphorylated counterparts; pyridoxine phosphate (PNP), and pyridoxamine phosphate (PMP).

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” or “engineered” or “non-naturally occurring” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

In principle the reaction of the present invention follows the below scheme:

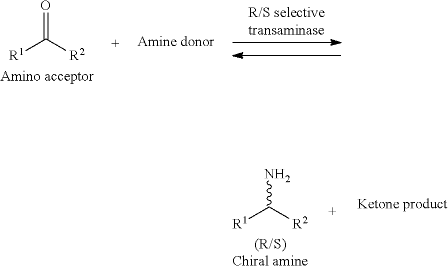

Thus, the present invention provides a process for the synthesis of optically active chiral amines by using at least one transaminase for the transamination of an amino group from an amino donor to a keto substrate acting as amino acceptor, thereby forming the desired product. Depending on the enantiopreference of the specific transaminase used, an optically active chiral amine is obtained. For instance the S-specific transaminase enzyme herein is capable of catalyzing the transfer of an amino group from an amino donor to a keto substrate, thereby forming S-specific chiral amine. Eventually, a R-specific transaminase enzyme catalyses the transfer of an amino group from an amino donor to a keto substrate, thereby forming R-specific chiral amine

In the context of the present invention the transaminase enzyme used comprises both naturally occurring (wild type) transaminase as well as non-naturally occurring engineered polypeptides generated by human manipulation. In general the transaminase enzyme described herein catalyses the transamination reaction by transfer of an amino group from an amino donor to an amino acceptor (ketone substrate). The products of this reaction are an amine product and an amino acceptor (ketone) byproduct.

In the context of the present invention an amino acceptor is a molecule capable of accepting an amino group transferred from an amino donor by a transaminase. In a particularly preferred embodiment of the present invention the amino acceptor contains a ketone functionality. The amino acceptor or keto substrate as described herein comprises a series of alpha hydroxy ketone compounds such as and not limited to those depicted in the formula 1, formula 2 and formula 3.

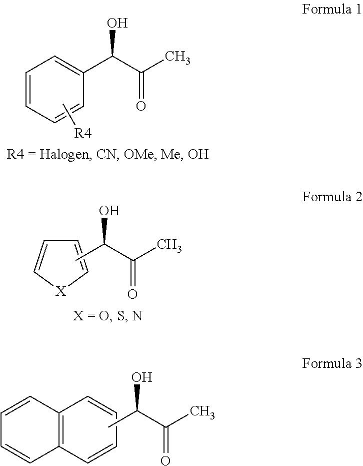

In the context of the present invention an amino donor is a molecule capable of providing an amino group to an amino acceptor or keto substrate using enzyme transaminase. In a particular preferred embodiment the amino donor is an amine or amino acid. Typical amino donors that can be used with the invention include chiral and achiral amino acids, and chiral and achiral amines. Amino donors that can be used with the invention include, by way of example and not limitation, isopropylamine (also termed 2-aminopropane), α-phenylethylamine (also termed 1-phenylethanamine), and its enantiomers (S)-1-phenylethanamine and (R)-1-phenylethanamine, 2-amino-4-phenylbutane, glycine, L-glutamic acid, L-glutamate, monosodium glutamate, L-alanine, D-alanine, D,L-alanine, L-aspartic acid, L-lysine, L-ornithine, β-alanine, taurine, n-octylamine, cyclohexylamine, 1,4-butanediamine, 1,6-hexanediamine, 6-aminohexanoic acid, 4-aminobutyric acid, tyramine, and benzyl amine, 2-aminobutane, 2-amino-1-butanol, 1-amino-1-(2-methoxy-5-fluorophenyl)ethane, 1-amino-1-phenylpropane, 1-amino-1-(4-hydroxyphenyl)propane, 1-amino-1-(4-bromophenyl)propane, 1-amino-1-(4-nitrophenyl)propane, 1-phenyl-2-aminopropane, 1-(3-trifluoromethylphenyl)-2-aminopropane, 2-aminopropanol, 1-amino-1-phenylbutane, 1-phenyl-2-aminobutane, 1-(2,5-dimethoxy-4-methylphenyl)-2-aminobutane, 1-phenyl-3-aminobutane, 1-(4-hydroxyphenyl)-3-aminobutane, 1-amino-2-methylcyclopentane, 1-amino-3-methylcyclopentane, 1-amino-2-methylcyclohexane, 1-amino-1-(2-naphthyl)ethane, 3-methylcyclopentylamine, 2-methylcyclopentylamine, 2-ethylcyclopentylamine, 2-methylcyclohexylamine, 3-methylcyclohexylamine, 1-aminotetralin, 2-aminotetralin, 2-amino-5-methoxytetralin, and 1-aminoindan, including both (R) and (S) single isomers where possible and including all possible salts of the amines.

In a particularly preferred embodiment the present invention therefore foresees reacting R-PAC (R-phenylacetylcarbinol) with an (S) or (R)-selective transaminase and an amino donor isopropylamine to obtain optically active (1R,2S) or (1R,2R) Norephedrine. Desamination of norephedrine to R-PAC was also investigated using pyruvate as the amine acceptor. The reaction rate is increasing with higher substrate concentration following the Michaelis-Menten enzyme kinetics.

Transamination of the substrates is carried out in a bioreactor using an aliquot of the enzyme with the substrate typically at a defined concentration. The reaction parameters such as pH, temperature, and mixing are maintained at levels that favor optimal biocatalytic activity and stability. A similar reaction can be done in continuous mode to recover the product on formation, to prevent reverse reaction.

The reaction could also be carried out in vivo by expression of the desired transaminase in the host, producing the alpha-hydroxy ketone by biotransformation. In the context of the present invention the desired host could be selected from the group of Saccharomyces sp., Pichia sp., Hansenula sp., Arthrobacter sp., Pseudomonas sp., E. coli sp. The described biotransformation process for the production of alpha hydroxy ketone takes place in the host cell wherein the host cell expresses the desired enzyme for carboligation. During the process of biotransformation reaction, the alpha-hydroxy ketone is produced in the host cell by external addition of aldehyde.

The alpha hydroxy ketone hence produced is then converted to corresponding amine in the presence of expressed transaminase enzyme. This could be a single pot in vivo process or a stage wise process involving in vivo/in vitro or a combination of in vivo and in vitro conversions in two or more stages involving single or multiple microorganisms, expressing enzymes of interest.

In order that this invention to be more fully understood the following preparative and testing examples are set forth. These examples are for the purpose of illustration only and are not to be construed as limiting the scope of the invention in any way.

Determination of diastereomeric purity using HPLC was carried out using the conditions given below:
Column: C₁₈ Nucleosil Machery Nagel, (250×4.6 mm) 5 μm,
Wavelength: 210 nm
Flow Rate: 1.0 ml/min
Run time: 20 min
Injection Volume: 20 μl
System Pressure: 12.00 to 14.00 MPa
Std Conc.: 0.1 mg/ml in mobile phase
Sample Conc.: 1.0 mg/ml in mobile phase

Mobile Phase: To 16 ml of 25% Tetramethyl ammonium hydroxide solution (MERCK make), HPLC grade water 500 ml is added, stirred well. Slowly 5 ml Ortho Phosphoric acid is added with stirring. Volume is made up to 1000 ml with HPLC grade water, mixed well. To 956 ml of above buffer, Methanol 40 ml & Tetra Hydrofuran 4 ml is added and stirred. The solution is filtered through 0.45 micron filter paper, sonicated, transferred in solvent reservoir.

In order that this invention be more fully understood, the following preparative and testing examples are set forth. These examples are for the purpose of illustration only and are not to be construed as limiting the scope of the invention in any way.

### EXAMPLE 1

### Screening of Transaminases

A reaction mixture comprising 5 mM R-PAC or 1-hydroxy-1-phenylpropan-2-one (99.5% w/v), 500 mM isopropylamine, 50 mM potassium phosphate at pH 7.4, 1 mM pyridoxal phosphate and Biomass of Category 1 bacteria (containing expressed enzyme S or R transaminase) 18 gm/L incubated under shaking at 30° C. overnight.
a) Expressed 5-transaminase (gives 1R, 2S product)
b) Expressed R-transaminase (gives 1R, 2R product)

>95% conversion with >98% de was obtained in either case for 2 transaminases under each category.

### EXAMPLE 2

### Production of optically active 2-amino-1-phenyl-1-propanol or R, S Norephedrine

A reaction mixture comprising of 25 mM R-PAC commercial, 50 mM Potassium Phosphate buffer, 1 mM pyridoxalphosphate, 500 mM isopropylamine and biomass (of Category 1 bacteria containing expressed S-transaminase) 9 gm/L is incubated at 30° C. under stirring for about 5 hours before adding another 25 mM of R-PAC commercial. 50 mM 1R, 2S Norephedrine base was obtained after a total incubation period of 26 hrs. The de% was 98%

Note: Commercial sample of R-PAC contains toluene, benzylalcohol and benzaldehyde in addition to about 25-35% R-PAC w/v. The residual activity of the enzyme is found to be intact up to 55-60° C. temperature exposure for 15 minutes and hence the transamination could be carried out at higher temperatures, with altered tolerance to impurities and increased reaction rate.

The reaction as described in the example follows the below scheme:

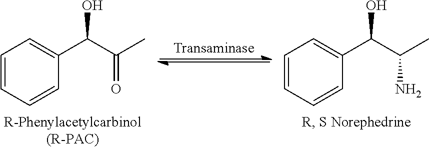

### EXAMPLE 3

### Production of optically active 2-amino-1-phenyl-1-propanol or R, S Norephedrine

### 1. Biomass Preparation

E. coli culture was scaled up to 1 L scale, by 2 stages of pre-culturing. The inoculum so obtained was used to inoculate the 10 fermentor containing about 6 L Medium.

### 2. Reaction

A reaction mixture comprising 0.5 gm (1%) R-PAC or 1-hydroxy-1-phenylpropan-2-one, 9 ml (0.96 M) isopropylamine, 30.5 ml 100 mM potassium phosphate buffer pH 7.4, 25 mg pyridoxal phosphate and biomass (containing enzyme transaminase) 3.75 gm wet weight, was incubated and kept for shaking at 30° C. for 48 hrs. 91% conversion was obtained with 98% diastereomeric excess.

Therefore, the present invention features an enzymatic method of producing optically active chiral amines by utilizing transaminase or aminotransaminase enzyme in the presence of defined amino donor.

1. A process for producing optically active chiral amine comprising:
a. providing an amino acceptor or keto substrate selected from a series of alpha hydroxy ketone and an amino donor;
b. reacting the keto substrate and the amino donor with a (R) or (S)-selective transaminase; and
c. finally obtaining the desired optically active chiral amine and a ketone by-product; wherein the process is carried out in a reaction mixture having a pH from approximately 6-8 for a reaction time of 12-48 hours in a temperature range from (25-35° C.)

2. The process according to claim 1, wherein the alpha hydroxy ketone is R-phenylacetylcarbinol.

3. The process according to claim 1, wherein the amino donor is selected from a group including amines or amino acids, in particular from isopropylamine (also termed 2-aminopropane), α-phenylethylamine (also termed 1-phenylethanamine), and its enantiomers (S)-1-phenylethanamine and (R)-1-phenylethanamine, 2-amino-4-phenylbutane, glycine, L-glutamic acid, L-glutamate, monosodium glutamate, L-alanine, D-alanine, D,L-alanine, L-aspartic acid, L-lysine, L-ornithine, β-alanine, taurine, n-octylamine, cyclohexylamine, 1,4-butanediamine, 1,6-hexanediamine, 6-aminohexanoic acid, 4-aminobutyric acid, tyramine, and benzyl amine, 2-aminobutane, 2-amino-1-butanol, 1-amino-1-(2-methoxy-5-fluorophenyl)ethane, 1-amino-1-phenylpropane, 1-amino-1-(4-hydroxyphenyl)propane, 1-amino-1-(4-bromophenyl)propane, 1-amino-1-(4-nitrophenyl)propane, 1-phenyl-2-aminopropane, 1-(3-trifluoromethylphenyl)-2-aminopropane, 2-aminopropanol, 1-amino-1-phenylbutane, 1-phenyl-2-aminobutane, 1-(2,5-dimethoxy-4-methylphenyl)-2-aminobutane, 1-phenyl-3-aminobutane, 1-(4-hydroxyphenyl)-3-aminobutane, 1-amino-2-methylcyclopentane, 1-amino-3-methylcyclopentane, 1-amino-2-methylcyclohexane, 1-amino-1-(2-naphthyl)ethane, 3-methylcyclopentylamine, 2-methylcyclopentylamine, 2-ethylcyclopentylamine, 2-methylcyclohexylamine, 3-methylcyclohexylamine, 1-aminotetralin, 2-aminotetralin, 2-amino-5-methoxytetralin, and 1-aminoindan.

4. The process according to claim 3, wherein the amino donor is isopropylamine.

5. The process according to claim 1, wherein the transaminase is from E. coli.

6. The process according to claim 1, wherein the optically active chiral amine is (1S, 2S) or (1R, 2R) Norephedrine.

7. The process according to claim 1, wherein the ketone-by-product is acetone.
